(19) United States
(12) Patent Application Publication
(10) Pub. No.: US 2006/0100678 A1
(43) Pub. Date: May 11, 2006

(54) Tanning device

(76) Inventor: Roger A. Brown, Dallas, TX (US)

Correspondence Address: LAW OFFICE OF PAUL W. FULBRIGHT, PLLC, 2003 J.J. PEARCE DRIVE, RICHARSON, TX, 75081-5447, US

(21) Appl. No.: 10/983,536

(22) Filed: Nov. 8, 2004

(51) Int. Cl. A61N 5/06

(52) U.S. Cl. 607/94

(57) ABSTRACT

A tanning device is disclosed. The tanning device comprises an elliptically-shaped conic reflector assembly comprising (1) a lighting element assembly emitting ultraviolet light waves, the lighting element assembly mounted at the vertex of the elliptically-shaped conic reflector assembly; (2) a reflector wall, the reflector wall comprising a reflective inner surface; and (3) at least one axis-directed reflective end portion, said reflector wall and said at least one axis-directed reflective end portion directing a plurality of said light waves axially towards and through a light filter mounted on a filter frame assembly positioned proximate to the base of said elliptically-shaped conic reflector assembly. The tanning device also comprises a fan assembly mounted proximately to the elliptically-shaped conic reflector assembly, so that the fan assembly can direct a portion of a cooling fluid (typically air) through at least one cooling fluid vent and at least one cooling fluid slot so as to effectively cool the device.

### BACKGROUND OF THE INVENTION

Tanning devices which endeavor to channel UV light feature numerous design constraints, and, as a result, prior art devices commonly suffer from at least one or more of the following problems: (a) inefficient channeling and use of ultraviolet light generated from a UV source; (b) ineffective cooling within the tanning device itself; and (c) ineffective cooling at the exterior of the device. For these reasons and more, there is a great demand for a tanning device which efficiently channels ultraviolet light while simultaneously transferring and discharging the heat inherently generated by these devices.

### BRIEF SUMMARY OF THE INVENTION

A tanning device is disclosed which features two main assemblies. The first is an elliptically shaped conic reflector assembly which channels light, and the second is a fan assembly which channels a cooling fluid (typically a gas such as air) so as to cool the device and the area surrounding it. These two assemblies are integrated in a novel and nonobvious manner in the instant disclosure.

The elliptically-shaped conic reflector assembly comprises a lighting element assembly which emits ultraviolet light waves and which is usually mounted at the vertex of the reflector assembly. It also comprises a reflector wall with a reflective inner surface (to channel light), and at least one (usually two) axis-directed reflective end portions which direct light waves axially towards and through a light filter mounted near the base of the conic reflector assembly.

As stated, the tanning device also comprises an important assembly, the fan assembly, designed to manage the high temperatures generated by the lighting element assembly. The fan assembly is mounted near the conic reflector assembly, and it is specifically designed to generate two distinct flows of air. First, it directs a portion of the cooling fluid through at least one cooling fluid vent so as to moderate the internal temperature in the interior of the conic reflector assembly. Second, it directs another portion of the cooling fluid through at least one cooling fluid slot so as to moderate the external temperature proximate to an exterior side of the light filter. These two flows of air, working together, effectively manage the temperature of the device.

### DESCRIPTION OF THE FIGURES

FIG. 1 provides a front perspective view of a tanning device 200.

FIG. 2 provides a rearward perspective view of the tanning device 200 of FIG. 1.

FIG. 3 provides a top view of the tanning device 200 of FIG. 1.

FIG. 4 provides a bottom view of the tanning device 200 of FIG. 1.

FIG. 5 provides a right-side view of the tanning device 200 of FIG. 1.

FIG. 6 provides a left-side view of the tanning device 200 of FIG. 1.

FIG. 7 provides a rear-side view of the tanning device 200 of FIG. 1.

FIG. 8 provides a front-side view of the tanning device 200 of FIG. 1.

FIG. 9 provides a perspective view of the bottom of the tanning device 200 of FIG. 1.

FIG. 10 provides an exploded perspective view of the tanning device 200 of FIG. 1.

FIG. 11 provides a top perspective view of the filter frame assembly 224

FIG. 12 provides a perspective view of a tanning bed 100 within which four (4) of the tanning devices 200 of FIG. 1 are used.

FIG. 13 provides a scale drawing revealing the inter-relationship of the assemblies and components of the tanning device 200 of FIG. 1.

FIG. 14 provides a scale drawing showing various aspects of the fan frame assembly of the tanning device 200 of FIG. 1.

FIG. 15 provides another scale drawing showing various aspects of the fan frame assembly of the tanning device 200 of FIG. 1.

FIG. 16 provides a scale drawing showing various aspects of the elliptically shaped conic reflector assembly (including, specifically, the axis-directed reflective end portion) of the tanning device 200 of FIG. 1.

### DETAILED DESCRIPTION

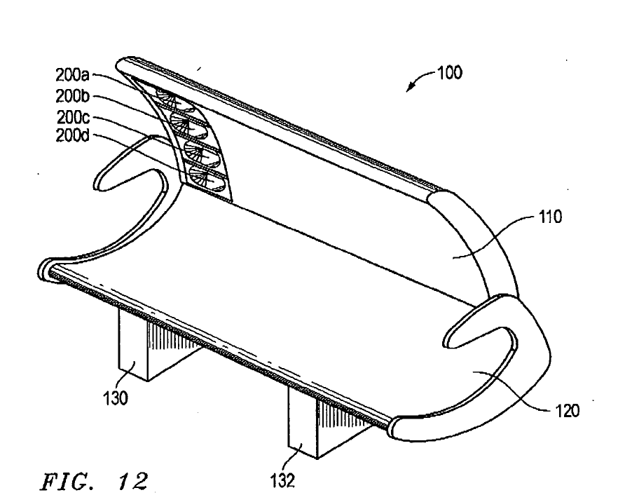

Turning now to the figures, and, specifically, to FIG. 12, a tanning bed 100 is depicted. The tanning bed features a bed portion 120 and a separate upper panel 110 which is raised and lowered over a person laying on the bed 100. Both the bed portion 120 and the upper panel 110 contain a plurality of ultraviolet lights (not shown) and controls specifically designed to operate the bed.

Also shown in FIG. 12 are the tanning devices 200 (in FIG. 12 specifically they are denominated tanning devices 200a, 200b, 200c, and 200d). In the instant application these devices 200 are specifically designed to manage the tanning process in the head area of the user, and they channel ultraviolet light for that purpose. In the case of the instant nonobvious design, the tanning devices 200 are mounted within the separate upper panel 110 with a space between the device 200 and an acrylic protective sheet of panel 110, the space providing a passageway for the circulation of a cooling fluid (typically air) which will carry away excess heat (as discussed further hereinbelow).

It is the design of these tanning devices 200 to which this disclosure is directed. It should also be noted that the principles utilized in the tanning devices 200 disclosed herein can be used to manufacture much larger tanning devices and/or whole tanning bed assemblies, as well as to channel ultraviolet light, other forms of light, and even other forms of electromagnetic radiation for other applications.

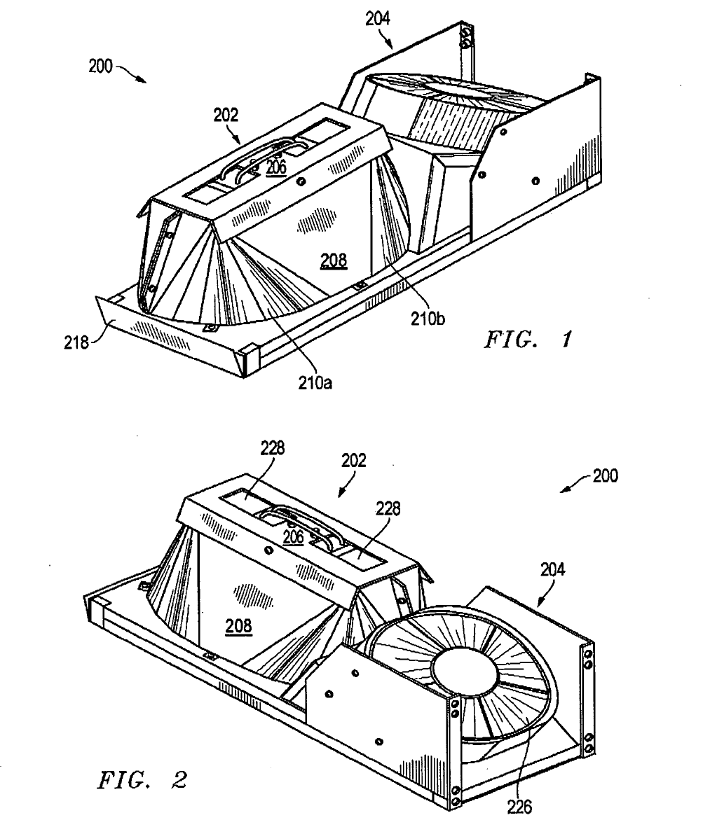

Returning, now, to FIG. 1, an electromagnetic ray—emitting device 200 (or light-emitting device 200), which can serve as an especially effective tanning device 200, is disclosed. This invention 200 (which, for convenience, will be referred to as a tanning device 200 herein) comprises two main subsystems: an elliptically-shaped conic reflector assembly 202 and a fan assembly 204.

The elliptically-shaped conic reflector assembly 202 comprises an electromagnetic ray—emitting element assembly 206 (which, in the case of the invention's use as a tanning device 200, is a lighting element assembly 206), a reflector wall 208, and at least one axis-directed reflective end portion 210 (in the preferred embodiment shown, there are two axis-directed reflective end portions 210a, 210b).

The electromagnetic ray—emitting element assembly 206 (which, for convenience, will be referred to as a lighting element assembly 206 herein) emits electromagnetic waves and, in the usual use of the invention 200 as a tanning device 200, is configured to emit ultraviolet light waves so as to facilitate carefully controlled tanning of the user.

As shown in the figures, the lighting element assembly 206 is mounted at the effective vertex of the elliptically-shaped conic reflector assembly 202.

The reflector wall 208, as its name implies, comprises an inner surface which is specifically designed to reflect and channel the rays emitted from assembly 206. In the case of the invention's use as a tanning device 200, the reflector wall 208 is commonly manufactured of polished aluminum; however, it can also be fashioned from a variety of other materials. For example, in ultraviolet tanning applications, the reflector wall 208 can also be manufactured from the trade material known as “AmBright.”

Finally, the elliptically-shaped conic reflector assembly 202 comprises at least one axis-directed reflective end portion 210. This end portion 210, like the reflector wall 208, is designed to reflect a plurality of light waves axially towards the base of the elliptically-shaped conic reflector assembly 202. At the base, a filter 212 may be mounted. The filter 212 is designed to alter the profile of electromagnetic and/or light waves emerging from the device 200; in the usual case of the invention's use as a tanning device 200, the filter 212 will effectually block the emergence of UVC and maximum UVB wavelengths to ensure safe, carefully controlled tanning by the user.

Turning, now, to FIG. 2, it will be observed that the tanning device 200 is optimally configured with a fan assembly 204. In brief, the fan assembly 204 exists to circulate a cooling fluid (in the usual case, a gas such as air) in and around the various tanning device 200 components so as to moderate tanning device 200 temperature. The temperatures observed in close proximity to the lighting element assembly 206 can be quite high, so the fan assembly 204 typically plays a key role in effecting convective cooling of the tanning device 200 through its generation of a stream of air which carries heat out of the system. It will be noted in this regard that the orientation of axis of the elliptically-shaped conic reflector assembly 202 is perpendicular to the base of the assembly 202 whereas the orientation of the axis of the fan assembly 204 is oriented at an angle to the base of assembly 202 (so as to encourage the flow of air towards and through the assembly 202 as described further hereinbelow).

It will also be observed that the lighting element assembly 206, which is mounted at the effective vertex of the elliptically-shaped conic reflector assembly 202, features one or more cooling fluid exit slots 228 to provide an additional exit for the transfer and discharge of heat.

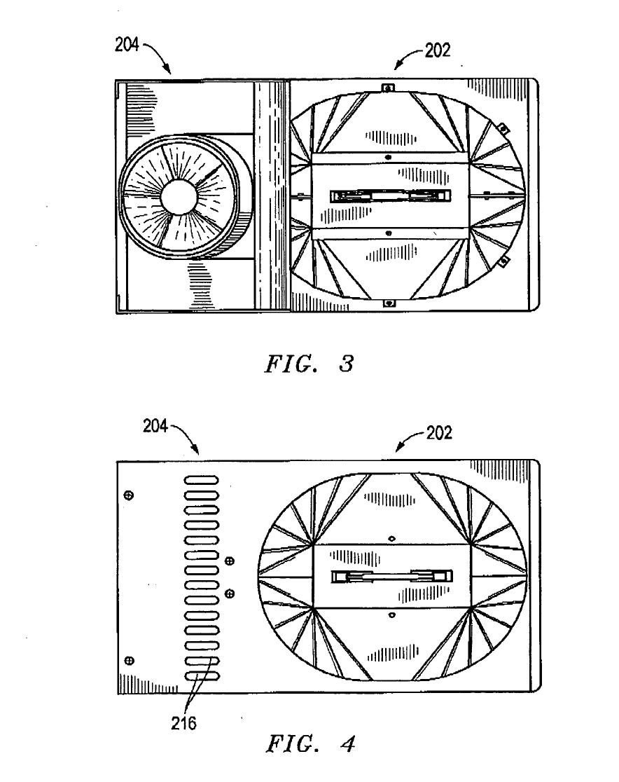

Turning, now, to FIG. 3 and FIG. 4, a top view of the tanning device 200 (see FIG. 3) and a bottom view of the tanning device 200 (see FIG. 4) is shown.

FIG. 3 (the top view) reveals the close proximity of the fan assembly 204 to the elliptically-shaped conic reflector assembly 202. This is so as to facilitate the high-speed convective circulation of a cooling fluid (usually a gas such as air) through the elliptically-shaped conic reflector assembly 202 by means of one or more cooling fluid vents 214. In the preferred embodiment, the cooling fluid vent 214 is the passageway defined between the fan assembly 204 and the filter frame assembly 224 which terminates in the cooling fluid exit flap 218 (as discussed further hereinbelow). Alternatively, the cooling fluid vent 214 could comprise an opening in the reflector wall 208 between the elliptically-shaped conic reflector assembly 202 and the fan assembly 204. Regardless of the design of the cooling fluid vent 214, it facilitates convective cooling which moderates the internal temperature in the interior of the elliptically-shaped conic reflector assembly 202 and at the interior side of the filter 212.

FIG. 4 (the bottom view) reveals that the fan assembly 204 not only directs a portion of cooling fluid through the one or more cooling fluid vents 214, but, in addition, directs another portion of the cooling fluid through one or more cooling fluid slots 216 so as to force convective air circulation outside the tanning device 200 and further carry heat away from the outside surface of the filter 212.

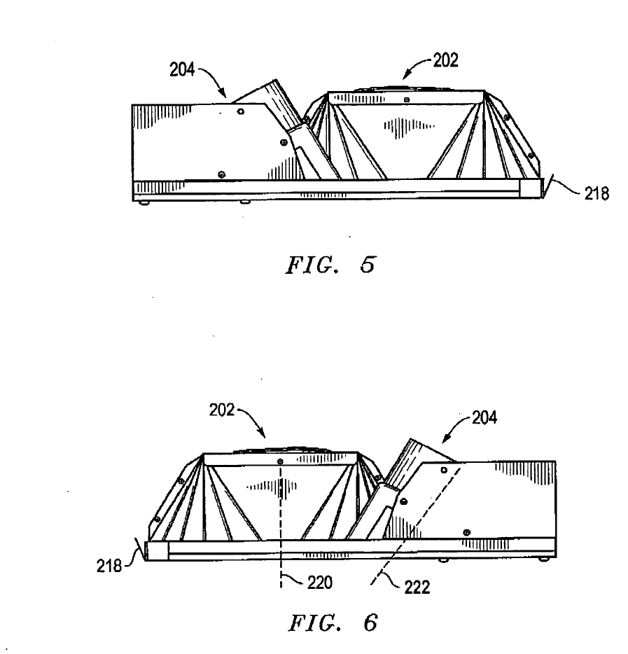

Turning, now, to FIG. 5 and FIG. 6, a right-side view of the tanning device 200 (see FIG. 5) and a left-side view of the tanning device 200 (see FIG. 6) is shown.

FIG. 5 (the right-side view) clearly reveals the presence of a cooling fluid exit flap 218 through which a portion of the cooling fluid exits the elliptically-shaped conic reflector assembly 202 and the tanning device 200.

FIG. 6 (the left-side view) also clearly demonstrates that the orientation of axis 220 of the elliptically-shaped conic reflector assembly 202 is perpendicular to the base of the assembly 202 whereas the orientation of the axis 222 of the fan assembly 204 is oriented at an angle to the base of assembly 202 (so as to encourage the flow of air towards and through the assembly 202).

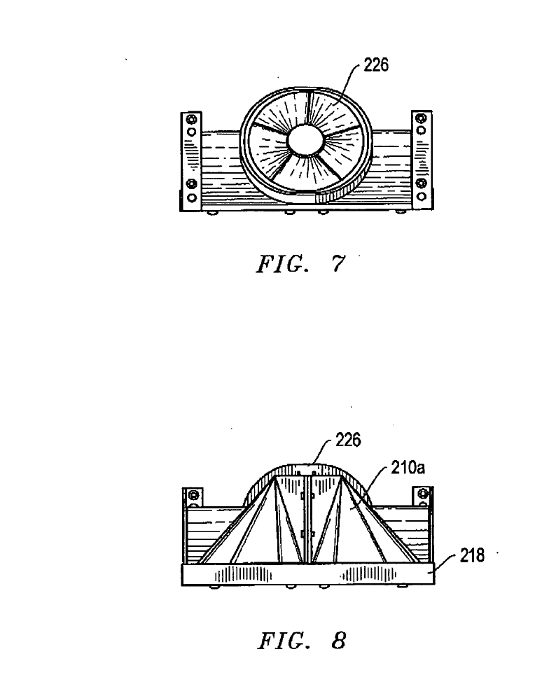

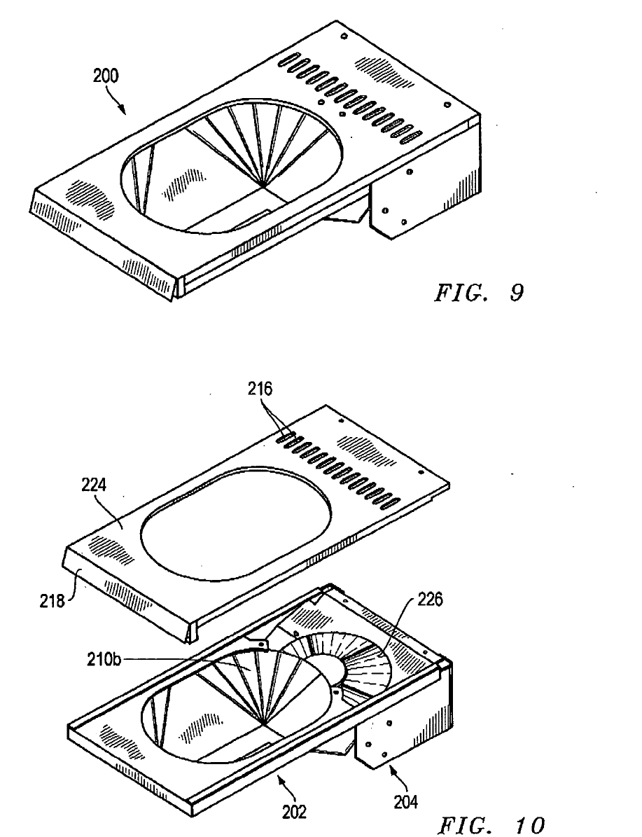

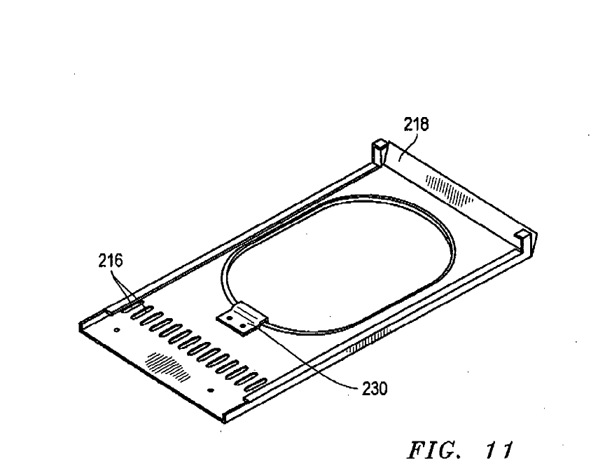

Turning, now, to FIG. 7 and FIG. 8, a rear-side view of the tanning device 200 (see FIG. 7) and a front-side view of the tanning device 200 (see FIG. 8) is shown.

FIG. 7 (the rear-side view) clearly reveals the presence of a cooling fluid circulating fan 226 which exists to force convective cooling of the entire tanning device 200. Reference to this FIG. 7 and the side view of preceding FIG. 6 reveals that the fan 226 is aligned so as to force the cooling fluid (typically air) straight down, and in alignment with, the long axis of the device 200.

FIG. 8 (the front-side view) also clearly demonstrates the alignment of the elliptically-shaped conic reflector assembly 202 vis-à-vis the fan assembly 204 and fan 226.

Turning, now, to FIG. 9 and FIG. 10, various details regarding the channeling of the light, as well as the management of the flow of the cooling fluid, become apparent.

FIG. 9 shows a perspective view of the bottom of the assembled tanning device 200.

FIG. 10 shows an exploded perspective view of the assembled tanning device 200 by splitting the filter frame assembly 224 from the combined elliptically shaped conic reflector assembly 202 and fan assembly 204. This FIG. 10 highlights the dual air flows generated by fan 226. Clearly, one portion of the cooling fluid flow passes through the cooling fluid slots 216 and then carries heat emanating from the exterior surface of the filter 212 out of the system. Another portion of the cooling fluid flow passes along the interior surface of the filter 212 and exits the system through the cooling fluid exit flap 218.

Turning, now, to FIG. 11, a top perspective view of the filter frame assembly 224 is shown. This view highlights the presence of a retaining clip that secures the filter into place.

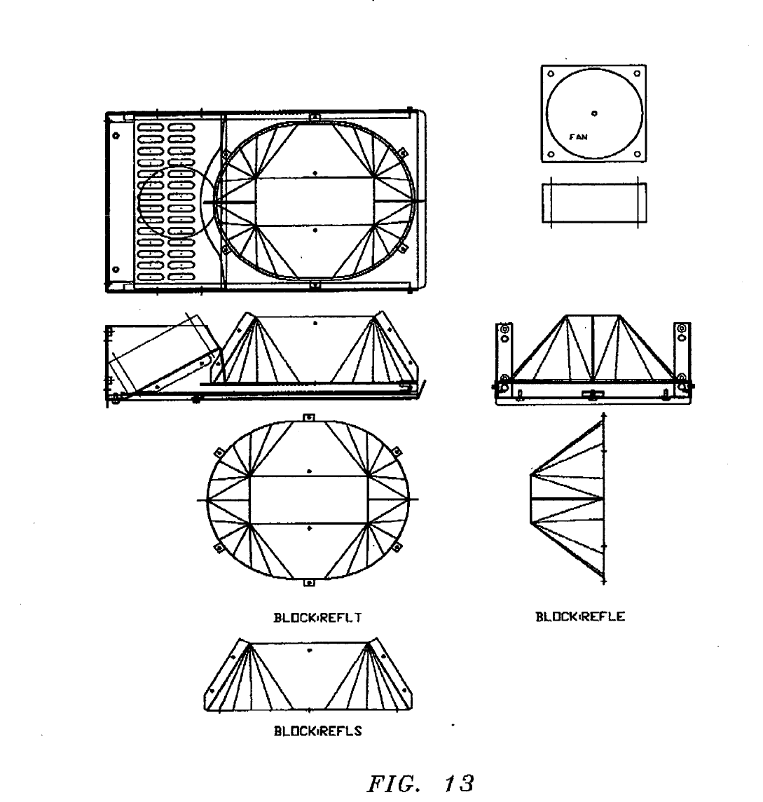

Turning, now, to FIG. 13, a scale drawing is provided which again depicts the inter-relationship of the assemblies and components of the tanning device 200. In particular, the positioning of the fan 226 is emphasized.

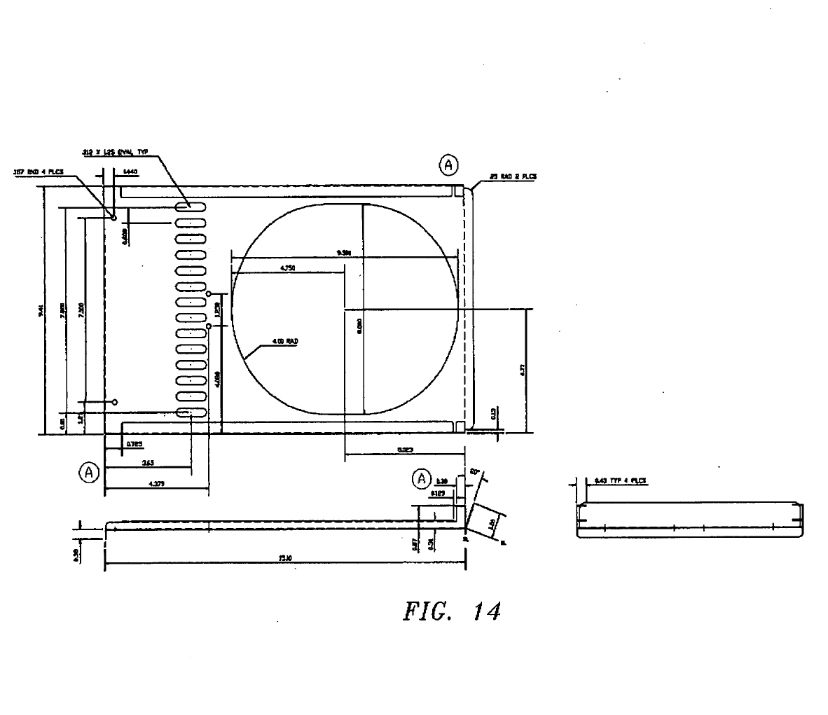

Turning, now, to FIG. 14 a scale drawing is provided which shows various aspects of the fan frame assembly 224 of the tanning device 200. In particular, various relative dimensions of a typical embodiment of the fan frame assembly 224 are depicted.

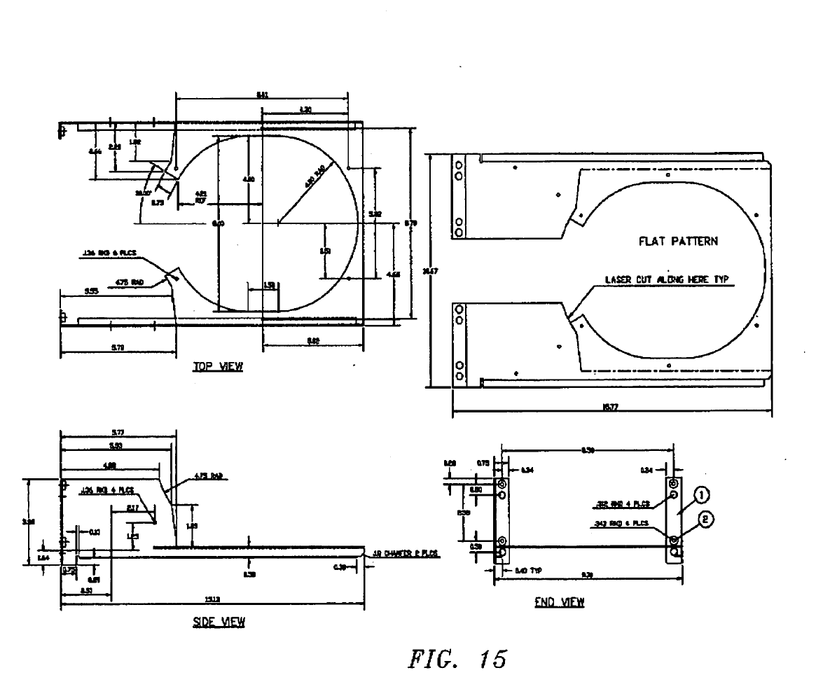

Turning, now, to FIG. 15, another scale drawing is provided which shows various aspects of the fan frame assembly 224 of the tanning device 200. This figure further clarifies various relative dimensions of a typical embodiment of the fan frame assembly 224.

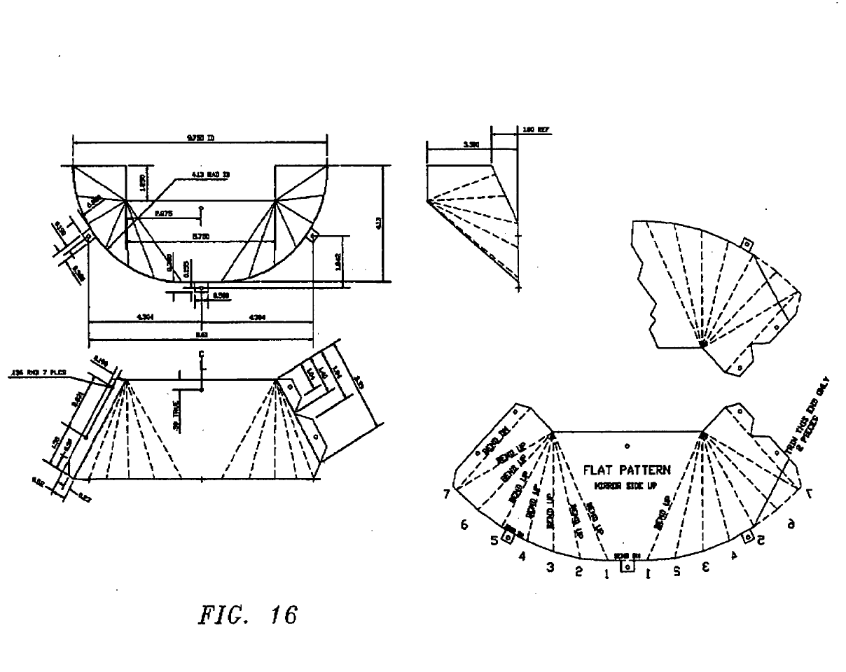

Turning, finally, to FIG. 16, a scale drawing is provided which shows various aspects of the elliptically shaped conic reflector assembly 202 of the tanning device 200. In particular, the relative dimensions of the axis-directed end portion 210 for a typical effective embodiment are depicted. Prior art devices, which typically incorporate inferior designs for the channeling of light, are not nearly as efficient in terms of the intensity of light effectively channeled to the user per unit of power.

The invention has been shown and described. However, the invention should not be viewed as being limited by the disclosure. Rather, the metes and bounds of the invention should be understood with reference to the claims.

I claim:

1. A tanning device comprising:
(a) an elliptically-shaped conic reflector assembly comprising (1) a lighting element assembly emitting ultraviolet light waves, said lighting element assembly mounted at the vertex of said elliptically-shaped conic reflector assembly; (2) a reflector wall, said reflector wall comprising a reflective inner surface; and (3) at least one axis-directed reflective end portion, said reflector wall and said at least one axis-directed reflective end portion directing a plurality of said light waves axially towards and through a light filter mounted on a filter frame assembly positioned proximate to the base of said elliptically-shaped conic reflector assembly; and
(b) a fan assembly mounted proximately to said elliptically-shaped conic reflector assembly, said fan assembly directing a portion of a cooling fluid through at least one cooling fluid vent defined by the proximity of said filter frame assembly to said elliptically shaped conic reflector assembly so as to moderate the internal temperature in the interior of said elliptically-shaped conic reflector assembly and at an interior side of said light filter, said fan assembly also directing another portion of said cooling fluid through at least one cooling fluid slot so as to moderate the external temperature proximate to an exterior side of said light filter.

2. The tanning device of claim 1 wherein the light filter filters an effective proportion of UVC and UVB light waves.

3. The tanning device of claim 1 wherein the cooling fluid is a gas.

4. The tanning device of claim 3 wherein the cooling fluid is air.

5. The tanning device of claim 1 wherein said reflective inner surface comprises polished aluminum.

6. The tanning device of claim 1 wherein said fan assembly further comprises a cooling fluid exit flap through which said portion of said cooling fluid exits said tanning device.

7. A light-emitting device comprising an elliptically-shaped conic reflector assembly, said elliptically-shaped conic reflector assembly comprising:
(1) a lighting element assembly emitting light waves, said lighting element assembly mounted at the vertex of said elliptically-shaped conic reflector assembly;
(2) a reflector wall, said reflector wall comprising a reflective inner surface; and
(3) at least one axis-directed reflective end portion, said reflector wall and said at least one axis-directed reflective end portion directing a plurality of said light waves axially towards and through a light filter mounted on a filter frame assembly positioned proximate to the base of said elliptically-shaped conic reflector assembly.

8. The light-emitting device of claim 7 wherein:
(a) said filter frame assembly's proximity to said elliptically-shaped conic reflector assembly defines at least one cooling fluid vent; and
(b) said light-emitting device further comprises a fan assembly mounted proximately to said elliptically-shaped conic reflector assembly, said fan assembly directing a portion of a cooling fluid through said at least one cooling fluid vent so as to moderate the internal temperature in the interior of said elliptically-shaped conic reflector assembly and at an interior side of said light filter.

9. The light-emitting device of claim 8 wherein the cooling fluid is a gas.

10. The light-emitting device of claim 8 wherein said filter frame assembly further comprises a cooling fluid exit flap through which said portion of said cooling fluid exits said light-emitting device.

11. The light-emitting device of claim 7 wherein said light-emitting device further comprises a fan assembly mounted proximately to said elliptically-shaped conic reflector assembly, said fan assembly directing a portion of a cooling fluid through at least one cooling fluid slot so as to moderate the external temperature proximate to an exterior side of said light filter.

12. The light-emitting device of claim 11 wherein the cooling fluid is a gas.

13. The light-emitting device of claim 12 wherein the cooling fluid is air.

14. The light-emitting device of claim 7 wherein said light waves are ultraviolet light waves.

15. The light-emitting device of claim 14 wherein the light filter filters an effective proportion of UVC and UVB light Waves.

16. The light-emitting device of claim 7 wherein said reflective inner surface comprises polished aluminum.

17. A light-emitting device comprising:
(a) an elliptically-shaped conic reflector assembly comprising (1) a lighting element assembly emitting light waves, said lighting element assembly mounted at the vertex of said elliptically-shaped conic reflector assembly; (2) a reflector wall, said reflector wall comprising a reflective inner surface; and (3) at least one axis-directed reflective end portion, said reflector wall and said at least one axis-directed reflective end portion directing a plurality of said light waves axially towards and through a light filter mounted on a filter frame assembly positioned proximate to the base of said elliptically-shaped conic reflector assembly; and
(b) a fan assembly mounted proximately to said elliptically-shaped conic reflector assembly, said fan assembly directing a portion of a cooling fluid through at least one cooling fluid vent defined by the proximity of said filter frame assembly to said elliptically shaped conic reflector assembly so as to moderate the internal temperature in the interior of said elliptically-shaped conic reflector assembly and at an interior side of said light filter, said fan assembly also directing another portion of said cooling fluid through at least one cooling fluid slot so as to moderate the external temperature proximate to an exterior side of said light filter.

18. The light-emitting device of claim 17 wherein said light waves are ultraviolet light waves.

19. The light-emitting device of claim 18 wherein the light filter filters an effective proportion of UVC and UVB light waves.

20. The light-emitting device of claim 17 wherein the cooling fluid is a gas.

21. The light-emitting device of claim 20 wherein the cooling fluid is air.

22. The light-emitting device of claim 17 wherein said reflective inner surface comprises polished aluminum.

23. The light-emitting device of claim 17 wherein said fan assembly further comprises a cooling fluid exit flap through which said portion of said cooling fluid exits said light-emitting device.

24. An elliptically-shaped conic reflector assembly comprising:
(a) a lighting element assembly emitting light waves, said lighting element assembly mounted at the vertex of said elliptically-shaped conic reflector assembly;
(b) a reflector wall, said reflector wall comprising a reflective inner surface; and
(c) at least one axis-directed reflective end portion, said reflector wall and said at least one axis-directed reflective end portion directing a plurality of said light waves axially towards and through a light filter mounted proximate to the base of said elliptically-shaped conic reflector assembly.

25. The elliptically-shaped conic reflector assembly of claim 24 wherein said light waves are ultraviolet light waves.

26. The elliptically-shaped conic reflector assembly of claim 25 wherein the light filter filters an effective proportion of UVC and UVB light waves.

27. The elliptically-shaped conic reflector assembly of claim 24 wherein said reflective inner surface comprises polished aluminum.
